(19) United States
(12) Patent Application Publication
(10) Pub. No.: US 2025/0073232 A1
(43) Pub. Date: Mar. 6, 2025

(54) COMBINATIONS OF LSD1 INHIBITORS FOR TREATING MYELOID CANCERS

(71) Applicant: ORYZON GENOMICS, S.A., Madrid (ES)

(72) Inventors: Tamara MAES, Cornella de Llobregat (ES); Natalia SACILOTTO, Cornella de Llobregat (ES)

(21) Appl. No.: 18/554,241

(22) PCT Filed: Mar. 21, 2022
(86) PCT No.: PCT/EP2022/057386

§ 371 Date: Oct. 6, 2023

(30) Foreign Application Priority Data
Apr. 8, 2021 (EP) 21382301.6
Jul. 23, 2021 (EP) 21382676.1

(51) Int. Cl. A61K 31/497; A61K 31/136; A61P 35/02

(52) U.S. Cl. CPC A61K 31/497; A61K 31/136; A61P 35/02

(57) ABSTRACT

The instant invention relates to combinations of LSD1 inhibitors (or pharmaceutically acceptable salts thereof) and gilteritinib (or a pharmaceutically acceptable salt thereof). The combinations are particularly useful for treating myeloid cancers, such as acute myeloid leukemia or myelodysplastic syndrome.

### FIELD

The present invention relates to combinations of LSD1 inhibitors and gilteritinib. The combinations are useful for treating myeloid cancers, particularly acute myeloid leukemia and myelodysplastic syndrome.

### BACKGROUND

Acute myeloid leukemia (AML) is an aggressive myeloid cancer that causes uncontrolled growth and accumulation of undifferentiated hematopoietic cells (blasts) leading to bone marrow failure, affectation of normal blood cell production, and ultimately, patient death months after diagnosis unless treated. AML is the most common acute leukemia in adults, and is primarily a disease of older people, with a median age at diagnosis of 68 years. As the population across the globe is growing and living longer, more patients are being diagnosed with AML each year. In fact, AML represents 1.1% of all new cancer diagnosis, and there were around 135,000 newly diagnosed cases of AML worldwide in 2019. Treatment of AML in people under 60 is standard with intense chemotherapy to induce remission enabling a subsequent bone marrow transplantation, the sole therapeutic approach considered curative in these patients. However, elderly patients or patients in poor health condition may not be able to tolerate this treatment, and their therapeutic options are limited to non-curative approaches such as low intensity chemotherapy, e.g. with azacitidine, alone or combined with venetoclax (the latter only approved in the USA), or to certain drugs aimed at specific sub-populations with certain mutations.

AML prognosis is poor, with survival ranging from 35-40% in adults aged <60 to as low as 5-15% in older patients. 25% of patients with AML are estimated to be refractory to treatment, and more than 50% are estimated to relapse to the current treatments. When patients in first-line treatment relapse or do not benefit from the therapy, they continue with second-line regimens, which are far less standardized and efficient; in fact, many of these patients are placed in clinical trials in view of lack of effective treatments. Even with active therapy, the prognosis of these relapsed/refractory (R/R) patients is very bad, showing a median survival of 6 months. A big portion of this R/R population (30 to 50% of all R/R AML cases) exhibit mutations in the FMS-like tyrosine kinase 3 (FLT3) gene, which are regarded as a marker of poor prognosis.

Myelodysplastic syndrome (MDS) is another type of myeloid cancer, in which differentiation of blood precursor cells is impaired and a significant increase in levels of apoptotic cell death occurs in bone marrow cells. Over time, about one-third of all MDS cases evolve to become AML. FLT3 mutations are also found in MDS.

Gilteritinib is a FLT3 inhibitor that is approved for the treatment of R/R AML patients with FLT3 mutations and is being evaluated in clinical trials in MDS. However, following gilteritinib therapy outcomes are still poor and only 21% of R/R AML patients show complete remission when treated with gilteritinib, and relapse-free survival is only around 4 months. There is thus a strong and unmet need for new improved therapeutic options for myeloid cancers, particularly AML and MDS, which address the problem of resistance and lack of responsiveness to current treatments. The present invention addresses these and other needs.

### SUMMARY OF THE INVENTION

The invention is based on the unexpected finding that the combination of an LSD1 inhibitor with gilteritinib, as described herein, exhibits outstanding activity in inhibiting the growth of myeloid cancer cells as compared to treatment with the LSD1 inhibitor alone or gilteritinib alone. Thus, the invention relates to novel combinations for treating myeloid malignancies such as AML and MDS by using LSD1 inhibitors in combination with gilteritinib.

Accordingly, the present invention provides a combination product comprising, in the same pharmaceutical formulation or in separate pharmaceutical formulations, an LSD1 inhibitor or a pharmaceutically acceptable salt thereof and gilteritinib or a pharmaceutically acceptable salt thereof.

The present invention further provides a pharmaceutical composition comprising an LSD1 inhibitor or a pharmaceutically acceptable salt thereof and gilteritinib or a pharmaceutically acceptable salt thereof, and one or more pharmaceutically acceptable excipients.

The present invention further provides an article of manufacture (or “kit”) comprising, in the same pharmaceutical formulation or in separate pharmaceutical formulations, an LSD1 inhibitor or a pharmaceutically acceptable salt thereof and gilteritinib or a pharmaceutically acceptable salt thereof.

The present invention further relates to the above-described combination product, the pharmaceutical composition or the article of manufacture, for use in therapy (or for use as a medicament/medicine). Thus, the invention particularly provides a combination product comprising, in the same pharmaceutical formulation or in separate pharmaceutical formulations, an LSD1 inhibitor or a pharmaceutically acceptable salt thereof and gilteritinib or a pharmaceutically acceptable salt thereof, for use in therapy.

The present invention further provides a combination product comprising, in the same pharmaceutical formulation or in separate pharmaceutical formulations, an LSD1 inhibitor or a pharmaceutically acceptable salt thereof and gilteritinib or a pharmaceutically acceptable salt thereof, for use in the treatment of a myeloid cancer which is preferably selected from acute myeloid leukemia and myelodysplastic syndrome.

The invention further provides a method for treating a myeloid cancer (which is preferably selected from acute myeloid leukemia and myelodysplastic syndrome) in a patient in need thereof, comprising administering to the patient a therapeutically effective amount of the above-described combination product, the pharmaceutical composition or the article of manufacture. In particular, the invention provides a method for treating a myeloid cancer (which is preferably selected from acute myeloid leukemia and myelodysplastic syndrome) in a patient in need thereof, comprising administering to the patient a therapeutically effective amount of a combination product comprising, in the same pharmaceutical formulation or in separate pharmaceutical formulations, an LSD1 inhibitor or a pharmaceutically acceptable salt thereof and gilteritinib or a pharmaceutically acceptable salt thereof.

The invention further provides a method for treating a myeloid cancer (which is preferably selected from acute myeloid leukemia and myelodysplastic syndrome) in a patient in need thereof, comprising administering to the patient a therapeutically effective amount of an LSD1 inhibitor, or a pharmaceutically acceptable salt thereof, and a therapeutically effective amount of gilteritinib, or a pharmaceutically acceptable salt thereof.

The invention further provides the use of a combination comprising an LSD1 inhibitor or a pharmaceutically acceptable salt thereof and gilteritinib or a pharmaceutically acceptable salt thereof for the manufacture of a medicament for the treatment of a myeloid cancer which is preferably selected from acute myeloid leukemia and myelodysplastic syndrome. The invention further provides the use of a combination comprising an LSD1 inhibitor or a pharmaceutically acceptable salt thereof and gilteritinib or a pharmaceutically acceptable salt thereof for the treatment of a myeloid cancer which is preferably selected from acute myeloid leukemia and myelodysplastic syndrome.

In preferred embodiments, the LSD1 inhibitor is iadademstat or a pharmaceutically acceptable salt thereof (e.g., iadademstat dihydrochloride).

### BRIEF DESCRIPTION OF THE DRAWINGS

FIG. 1 shows the plate organization for the matrix assays used to determine synergistic effects of the combinations of the invention, as described in Example 1.

### DETAILED DESCRIPTION OF THE INVENTION

As indicated above, the present invention is based on the surprising discovery that LSD1 inhibitors and gilteritinib, as described herein, can be used in combination to treat myeloid malignancies, with superior anticancer efficacy than attained by treatment with the LSD1 inhibitor alone or gilteritinib alone, as explained in more detail below and in the Examples.

In accordance with the present invention, an “LSD1 inhibitor” refers to a compound that reduces, decreases, blocks or inhibits the gene expression, activity or function of LSD1. Examples thereof are provided below under the heading “LSD1 inhibitors”. A preferred LSD1 inhibitor is iadademstat or a pharmaceutically acceptable salt thereof (e.g., iadademstat dihydrochloride).

In detail, the present invention provides a combination product comprising, in the same pharmaceutical formulation or in separate pharmaceutical formulations, an LSD1 inhibitor or a pharmaceutically acceptable salt thereof and gilteritinib or a pharmaceutically acceptable salt thereof. The LSD1 inhibitor (or the pharmaceutically acceptable salt thereof) and gilteritinib (or the pharmaceutically acceptable salt thereof) may thus be present in a single pharmaceutical formulation (i.e., in the same pharmaceutical formulation), or they may each be provided in a distinct (separate) pharmaceutical formulation.

The present invention further provides a pharmaceutical composition comprising an LSD1 inhibitor or a pharmaceutically acceptable salt thereof and gilteritinib or a pharmaceutically acceptable salt thereof, and one or more pharmaceutically acceptable excipients.

The present invention further provides an article of manufacture comprising, in the same pharmaceutical formulation or in separate pharmaceutical formulations, an LSD1 inhibitor or a pharmaceutically acceptable salt thereof and gilteritinib or a pharmaceutically acceptable salt thereof.

The present invention further provides a combination product comprising, in the same pharmaceutical formulation or in separate pharmaceutical formulations, an LSD1 inhibitor or a pharmaceutically acceptable salt thereof and gilteritinib or a pharmaceutically acceptable salt thereof, for use in therapy (or for use as a medicament/medicine). The invention likewise relates to the above-described pharmaceutical composition or the article of manufacture, for use in therapy (or for use as a medicament/medicine).

The present invention furthermore provides the above-described combination product, the pharmaceutical composition or the article of manufacture, for use in the treatment of cancer, preferably for use in the treatment of a myeloid cancer, such as acute myeloid leukemia or myelodysplastic syndrome.

The present invention thus provides, in particular, a combination product comprising, in the same pharmaceutical formulation or in separate pharmaceutical formulations, an LSD1 inhibitor or a pharmaceutically acceptable salt thereof and gilteritinib or a pharmaceutically acceptable salt thereof, for use in the treatment of a myeloid cancer which is preferably selected from acute myeloid leukemia and myelodysplastic syndrome.

The invention further provides an LSD1 inhibitor or a pharmaceutically acceptable salt thereof, for use in the treatment of a myeloid cancer (which is preferably selected from acute myeloid leukemia and myelodysplastic syndrome), wherein the LSD1 inhibitor or the pharmaceutically acceptable salt thereof is for use in combination with gilteritinib or a pharmaceutically acceptable salt thereof. Accordingly, the invention provides an LSD1 inhibitor or a pharmaceutically acceptable salt thereof, for use in the treatment of a myeloid cancer (which is preferably selected from acute myeloid leukemia and myelodysplastic syndrome), wherein the LSD1 inhibitor or the pharmaceutically acceptable salt thereof is administered in combination with gilteritinib or a pharmaceutically acceptable salt thereof. The LSD1 inhibitor (or the pharmaceutically acceptable salt thereof) and gilteritinib (or the pharmaceutically acceptable salt thereof) may be provided in the same pharmaceutical formulation, or they may be provided in separate pharmaceutical formulations. The invention further provides gilteritinib or a pharmaceutically acceptable salt thereof, for use in the treatment of a myeloid cancer (which is preferably selected from acute myeloid leukemia and myelodysplastic syndrome), wherein gilteritinib or the pharmaceutically acceptable salt thereof is for use in combination with an LSD1 inhibitor or a pharmaceutically acceptable salt thereof. Accordingly, the invention provides gilteritinib or a pharmaceutically acceptable salt thereof, for use in the treatment of a myeloid cancer (which is preferably selected from acute myeloid leukemia and myelodysplastic syndrome), wherein said gilteritinib or the pharmaceutically acceptable salt thereof is administered in combination with an LSD1 inhibitor or a pharmaceutically acceptable salt thereof. The gilteritinib (or the pharmaceutically acceptable salt thereof) and the LSD1 inhibitor (or the pharmaceutically acceptable salt thereof) may be provided in the same pharmaceutical formulation, or they may be provided in separate pharmaceutical formulations.

The invention further provides a method for treating cancer, particularly a myeloid cancer (which is preferably selected from acute myeloid leukemia and myelodysplastic syndrome), in a patient in need thereof, comprising administering to the patient a therapeutically effective amount of the above-described combination product, the pharmaceutical composition or the article of manufacture.

In particular, the invention provides a method for treating a myeloid cancer (which is preferably selected from acute myeloid leukemia and myelodysplastic syndrome) in a patient in need thereof, comprising administering to the patient a therapeutically effective amount of a combination product comprising, in the same pharmaceutical formulation or in separate pharmaceutical formulations, an LSD1 inhibitor or a pharmaceutically acceptable salt thereof and gilteritinib or a pharmaceutically acceptable salt thereof.

The invention further provides a method for treating a myeloid cancer (which is preferably selected from acute myeloid leukemia and myelodysplastic syndrome) in a patient in need thereof, comprising administering to the patient a therapeutically effective amount of an LSD1 inhibitor, or a pharmaceutically acceptable salt thereof, and a therapeutically effective amount of gilteritinib, or a pharmaceutically acceptable salt thereof. The LSD1 inhibitor (or the pharmaceutically acceptable salt thereof) and gilteritinib (or the pharmaceutically acceptable salt thereof) may be provided/administered in the same pharmaceutical formulation, or they may be provided/administered in separate pharmaceutical formulations.

The invention further provides the use of a combination comprising an LSD1 inhibitor or a pharmaceutically acceptable salt thereof and gilteritinib or a pharmaceutically acceptable salt thereof for the manufacture of a medicament for the treatment of a myeloid cancer which is preferably selected from acute myeloid leukemia and myelodysplastic syndrome. The invention further provides the use of an LSD1 inhibitor or a pharmaceutically acceptable salt thereof in combination with gilteritinib or a pharmaceutically acceptable salt thereof for the manufacture of a medicament comprising, in the same pharmaceutical formulation or in separate pharmaceutical formulations, said LSD1 inhibitor or the pharmaceutically acceptable salt thereof and said gilteritinib or the pharmaceutically acceptable salt thereof, for the treatment of a myeloid cancer which is preferably selected from acute myeloid leukemia and myelodysplastic syndrome. The invention further provides the use of an LSD1 inhibitor or a pharmaceutically acceptable salt thereof in combination with gilteritinib or a pharmaceutically acceptable salt thereof for the manufacture of a medicament for the treatment of a myeloid cancer (which is preferably selected from acute myeloid leukemia and myelodysplastic syndrome), wherein the medicament comprises the LSD1 inhibitor or the pharmaceutically acceptable salt thereof and gilteritinib or the pharmaceutically acceptable salt thereof in the same pharmaceutical formulation or in separate pharmaceutical formulations.

The invention further provides the use of an LSD1 inhibitor or a pharmaceutically acceptable salt thereof for the manufacture of a medicament for the treatment of a myeloid cancer (which is preferably selected from acute myeloid leukemia and myelodysplastic syndrome), to be used in combination with gilteritinib or a pharmaceutically acceptable salt thereof.

The invention further provides the use of an LSD1 inhibitor or a pharmaceutically acceptable salt thereof for the manufacture of a medicament for the treatment of a myeloid cancer (which is preferably selected from acute myeloid leukemia and myelodysplastic syndrome) in combination with gilteritinib or a pharmaceutically acceptable salt thereof. The invention further provides the use of an LSD1 inhibitor or a pharmaceutically acceptable salt thereof for the manufacture of a medicament for the treatment of a myeloid cancer (which is preferably selected from acute myeloid leukemia and myelodysplastic syndrome), wherein said medicament is prepared for combined use (or for use in combination) with gilteritinib or a pharmaceutically acceptable salt thereof.

The invention further provides the use of gilteritinib or a pharmaceutically acceptable salt thereof for the manufacture of a medicament for the treatment of a myeloid cancer (which is preferably selected from acute myeloid leukemia and myelodysplastic syndrome), to be used in combination with an LSD1 inhibitor or a pharmaceutically acceptable salt thereof.

The invention further provides the use of gilteritinib or a pharmaceutically acceptable salt thereof for the manufacture of a medicament for the treatment of a myeloid cancer (which is preferably selected from acute myeloid leukemia and myelodysplastic syndrome) in combination with an LSD1 inhibitor or a pharmaceutically acceptable salt thereof.

The invention further provides the use of gilteritinib or a pharmaceutically acceptable salt thereof for the manufacture of a medicament for the treatment of a myeloid cancer (which is preferably selected from acute myeloid leukemia and myelodysplastic syndrome), wherein said medicament is prepared for combined use (or for use in combination) with an LSD1 inhibitor or a pharmaceutically acceptable salt thereof.

The invention further provides the use of a combination comprising an LSD1 inhibitor or a pharmaceutically acceptable salt thereof and gilteritinib or a pharmaceutically acceptable salt thereof for the treatment of a myeloid cancer which is preferably selected from acute myeloid leukemia and myelodysplastic syndrome.

The invention further provides the use of an LSD1 inhibitor or a pharmaceutically acceptable salt thereof in combination with gilteritinib or a pharmaceutically acceptable salt thereof for the treatment of a myeloid cancer (which is preferably selected from acute myeloid leukemia and myelodysplastic syndrome), wherein said LSD1 inhibitor or the pharmaceutically acceptable salt thereof and said gilteritinib or the pharmaceutically acceptable salt thereof are provided in the same pharmaceutical formulation or in separate pharmaceutical formulations.

The invention further provides the use of an LSD1 inhibitor or a pharmaceutically acceptable salt thereof for the treatment of a myeloid cancer (which is preferably selected from acute myeloid leukemia and myelodysplastic syndrome), to be used in combination with gilteritinib or a pharmaceutically acceptable salt thereof.

The invention further provides the use of an LSD1 inhibitor or a pharmaceutically acceptable salt thereof for the treatment of a myeloid cancer (which is preferably selected from acute myeloid leukemia and myelodysplastic syndrome) in combination with gilteritinib or a pharmaceutically acceptable salt thereof.

The invention further provides the use of an LSD1 inhibitor or a pharmaceutically acceptable salt thereof for the treatment of a myeloid cancer (which is preferably selected from acute myeloid leukemia and myelodysplastic syndrome), wherein the LSD1 inhibitor or the pharmaceutically acceptable salt thereof is administered in combination with gilteritinib or a pharmaceutically acceptable salt thereof.

The invention further provides the use of gilteritinib or a pharmaceutically acceptable salt thereof for the treatment of a myeloid cancer (which is preferably selected from acute myeloid leukemia and myelodysplastic syndrome), to be used in combination with an LSD1 inhibitor or a pharmaceutically acceptable salt thereof.

The invention further provides the use of gilteritinib or a pharmaceutically acceptable salt thereof for the treatment of a myeloid cancer (which is preferably selected from acute myeloid leukemia and myelodysplastic syndrome) in combination with an LSD1 inhibitor or a pharmaceutically acceptable salt thereof.

The invention further provides the use of gilteritinib or a pharmaceutically acceptable salt thereof for the treatment of a myeloid cancer (which is preferably selected from acute myeloid leukemia and myelodysplastic syndrome), wherein said gilteritinib or the pharmaceutically acceptable salt thereof is administered in combination with an LSD1 inhibitor or a pharmaceutically acceptable salt thereof.

In the methods and uses according to the invention, the patient is a human being or an animal (e.g., a non-human mammal), preferably a human being.

In some embodiments, the LSD1 inhibitor is a small molecule.

In some embodiments, the LSD1 inhibitor is selected from the group consisting of iadademstat, pulrodemstat (CC-90011), bomedemstat, seclidemstat, 1-((4-(methoxymethyl)-4-(((1R,2S)-2-phenylcyclopropylamino) methyl) piperidin-1-yl)methyl)cyclobutanecarboxylic acid, 3-(cyanomethyl)-3-(4-{[(1R,2S)-2-phenylcyclopropyl]amino}piperidin-1-yl)azetidine-1-sulfonamide, and pharmaceutically acceptable salts thereof (i.e., pharmaceutically acceptable salts of any one of the aforementioned agents).

In some embodiments, the LSD1 inhibitor is selected from the group consisting of iadademstat, pulrodemstat (CC-90011), bomedemstat, and pharmaceutically acceptable salts thereof.

In some embodiments, the LSD1 inhibitor is pulrodemstat (CC-90011), or a pharmaceutically acceptable salt thereof.

In some embodiments, the LSD1 inhibitor is bomedemstat, or a pharmaceutically acceptable salt thereof.

Preferably, the LSD1 inhibitor is iadademstat or a pharmaceutically acceptable salt thereof. In some embodiments, the LSD1 inhibitor is iadademstat dihydrochloride.

In some embodiments, the myeloid cancer is acute myeloid leukemia.

In some embodiments, the acute myeloid leukemia is relapsed or refractory acute myeloid leukemia.

In some embodiments, the acute myeloid leukemia is relapsed acute myeloid leukemia.

In some embodiments, the acute myeloid leukemia is refractory acute myeloid leukemia.

In some embodiments, the acute myeloid leukemia is acute myeloid leukemia with a genetic, epigenetic or post-transcriptional alteration affecting (e.g., increasing) FLT3 expression and/or FLT3 activity. In particular, the acute myeloid leukemia may be acute myeloid leukemia with a FLT3 mutation and/or a genetic, epigenetic or post-transcriptional alteration affecting (e.g., increasing) FLT3 expression and/or FLT3 activity. In some embodiments, the acute myeloid leukemia is acute myeloid leukemia with a FLT3 mutation and/or a genetic, epigenetic or post-transcriptional alteration resulting in an increased FLT3 expression level or an increased FLT3 activity, wherein said increased FLT3 expression level or said increased FLT3 activity leads to uncontrolled cellular proliferation.

In some embodiments, the acute myeloid leukemia is acute myeloid leukemia with a FLT3 mutation.

In some embodiments, the acute myeloid leukemia is relapsed or refractory acute myeloid leukemia with a genetic, epigenetic or post-transcriptional alteration affecting (e.g., increasing) FLT3 expression and/or FLT3 activity. For example, the acute myeloid leukemia may be relapsed or refractory acute myeloid leukemia with a FLT3 mutation and/or a genetic, epigenetic or post-transcriptional alteration affecting (e.g., increasing) FLT3 expression and/or FLT3 activity.

In some embodiments, the acute myeloid leukemia is relapsed or refractory acute myeloid leukemia with a FLT3 mutation.

In some embodiments, the acute myeloid leukemia is relapsed acute myeloid leukemia with a genetic, epigenetic or post-transcriptional alteration affecting (e.g., increasing) FLT3 expression and/or FLT3 activity. For example, the acute myeloid leukemia may be relapsed acute myeloid leukemia with a FLT3 mutation and/or a genetic, epigenetic or post-transcriptional alteration affecting (e.g., increasing) FLT3 expression and/or FLT3 activity.

In some embodiments, the acute myeloid leukemia is relapsed acute myeloid leukemia with a FLT3 mutation.

In some embodiments, the acute myeloid leukemia is refractory acute myeloid leukemia with a genetic, epigenetic or post-transcriptional alteration affecting (e.g., increasing) FLT3 expression and/or FLT3 activity. For example, the acute myeloid leukemia may be refractory acute myeloid leukemia with a FLT3 mutation and/or a genetic, epigenetic or post-transcriptional alteration affecting (e.g., increasing) FLT3 expression and/or FLT3 activity.

In some embodiments, the acute myeloid leukemia is refractory acute myeloid leukemia with a FLT3 mutation.

In some embodiments, the FLT3 mutation is an activating FLT3 mutation, particularly a mutation resulting in ligand-independent FLT3 dimerization and constitutive activation of FLT3.

In some embodiments, the FLT3 mutation is an internal tandem duplication mutation in the juxtamembrane domain (FLT3-ITD) or a point mutation or deletion in the tyrosine kinase domain (FLT3-TKD). In some embodiments, the FLT3 mutation is FLT3-ITD. In some embodiments, the FLT3 mutation is FLT3-TKD. In some embodiments, the FLT3 mutation is FLT3-ITD and FLT3-TKD.

In some embodiments, the FLT3 mutation is a mutation in the tyrosine kinase domain (FLT3-TKD), particularly a point mutation (e.g., a nucleotide substitution) affecting (or involving) the aspartic acid residue in position 835 (D835) of wild-type FLT3 or a deletion of D835, and/or a point mutation (e.g., a nucleotide substitution) affecting (or involving) the isoleucine residue in position 836 (1836) of wild-type FLT3 or a deletion of 1836. Accordingly, the FLT3 mutation may be (or may comprise), for example, a D835 mutation, an 1836 mutation, or a D835/1836 mutation. In particular, a D835 mutation may be, e.g., a D835Y mutation (i.e., an FLT3 mutation wherein the aspartic acid (D) residue in position 835 (D835) is replaced/substituted by a tyrosine (Y) residue), a D835V mutation, a D835H mutation, a D835G mutation, a D835N mutation, or a deletion of D835. In some embodiments, the FLT3 mutation is (or comprises) a D835Y mutation. Moreover, the FLT3 mutation may also be (or may comprise) a point mutation affecting/involving the tyrosine residue in position 842 (Y842) of wild-type FLT3 or a deletion of Y842, a point mutation affecting/involving the lysine residue in position 663 (K663) of wild-type FLT3 or a deletion of K663, and/or a point mutation affecting/involving the valine residue in position 592 (V592) of wild-type FLT3 or a deletion of V592, such as, e.g., a Y842C mutation, a K663Q mutation, or a V592A mutation, or any combination thereof.

In some embodiments, the LSD1 inhibitor (or the pharmaceutically acceptable salt thereof) and gilteritinib (or the pharmaceutically acceptable salt thereof) are used as second-line treatment or as third-line treatment of relapsed or refractory acute myeloid leukemia.

In some embodiments, the myeloid cancer is myelodysplastic syndrome. In some embodiments, the myeloid cancer is myelodysplastic syndrome with a genetic, epigenetic or post-transcriptional alteration affecting (e.g., increasing) FLT3 expression and/or FLT3 activity. In particular, the myeloid cancer may be myelodysplastic syndrome with a FLT3 mutation and/or a genetic, epigenetic or post-transcriptional alteration affecting (e.g., increasing) FLT3 expression and/or FLT3 activity. In some embodiments, the myeloid cancer is myelodysplastic syndrome with a FLT3 mutation (e.g., any of the exemplary FLT3 mutations described above) and/or a genetic, epigenetic or post-transcriptional alteration resulting in an increased FLT3 expression level or an increased FLT3 activity, wherein said increased FLT3 expression level or said increased FLT3 activity leads to uncontrolled cellular proliferation. In some embodiments, the myeloid cancer is myelodysplastic syndrome with a FLT3 mutation (e.g., any of the exemplary FLT3 mutations described above).

In some embodiments, the LSD1 inhibitor (or the pharmaceutically acceptable salt thereof) and gilteritinib (or the pharmaceutically acceptable salt thereof) are administered as separate pharmaceutical formulations. To this end, the LSD1 inhibitor (or the pharmaceutically acceptable salt thereof) and gilteritinib (or the pharmaceutically acceptable salt thereof) are provided as separate pharmaceutical formulations.

Preferably, the LSD1 inhibitor, such as iadademstat (or a pharmaceutically acceptable salt thereof), is administered orally. Exemplary formulations which can be administered via peroral ingestion are described in more detail further below.

Preferably, gilteritinib (or a pharmaceutically acceptable salt thereof) is administered orally. Exemplary formulations which can be administered via peroral ingestion are described in more detail further below.

As illustrated in the Examples, it has been unexpectedly found in the context of the present invention that the combination of an LSD1 inhibitor with gilteritinib exhibits strong synergistic effects in inhibiting the growth of myeloid cancer such as AML. As explained in Example 1, treatment with the combination of an LSD1 inhibitor and gilteritinib, using two structurally unrelated, dissimilar LSD1 inhibitors, namely iadademstat, an irreversible, cyclopropylamine-based LSD1 inhibitor, and pulrodemstat (CC-90011), a reversible, non-cyclopropylamine-based LSD1 inhibitor, exhibited synergistic effects in inhibiting the growth of AML cell lines of different genetic backgrounds. Strong synergy for the combinations iadademstat plus gilteritinib and pulrodemstat (CC-90011) plus gilteritinib was observed in the FLT3-mutant AML cell lines, MOLM-13 and MV(4; 11). Remarkably, synergy was also observed in FLT3-wild-type (i.e. without FLT3 mutations) AML cell lines OCI-AML3 and TF-1a, which are resistant or poorly responsive to treatment with gilteritinib or other current AML therapies like venetoclax, as illustrated in Example 1. These findings show that the combination of an LSD1 inhibitor, such as iadademstat (or a pharmaceutically acceptable salt thereof), and gilteritinib (or a pharmaceutically acceptable salt thereof) is particularly useful to treat AML and other myeloid cancers such as MDS, with or without FLT3 mutations, even in those patients that are refractory to other treatments or which relapse.

The therapeutic effects of the combination of an LSD1 inhibitor and gilteritinib for the treatment of myeloid cancers such as AML can be further confirmed in additional in vitro or in vivo experiments, as well as in clinical trials in humans, which can be readily set up by those skilled in the art of drug development.

### LSD1 Inhibitors

As indicated earlier, as used herein an “LSD1 inhibitor” means a compound that reduces, decreases, blocks or inhibits the gene expression, activity or function of LSD1. Compounds which act as inhibitors of LSD1 are known in the art.

Any molecule acting as a LSD1 inhibitor can in principle be used in the context of the combinations, methods and uses according to the invention. Preferably, the LSD1 inhibitor is a small molecule. Irreversible and reversible LSD1 inhibitors have been described and can be used in the context of the present invention, as shown by the results described in the Examples herein below, using combinations of gilteritinib with both irreversible and reversible LSD1 inhibitors. Prototypical irreversible LSD1 inhibitors are cyclopropylamine-based compounds like iadademstat, one of the LSD1 inhibitors used in the Examples herein. A representative example of a reversible LSD1 inhibitor is the compound pulrodemstat (CC-90011), which has also been used in the Examples herein. Preferably, the LSD1 inhibitor is a selective LSD1 inhibitor; as used herein, a “selective LSD1 inhibitor” means an LSD1 inhibitor which exhibits a selectivity of at least 10-fold for LSD1 over other FAD-dependent monoamine oxidases, particularly MAO-A and MAO-B. An exemplary list of small molecule LSD1 inhibitors is provided in the Table below:

| | | International Non- |
|---|---|---|
| | Generic | proprietary Name |
| Chemical name | name/Code | (INN) |
| (trans)-N1-((1R,2S)-2-phenylcyclopropyl)cyclohexane-1,4-diamine | ORY-1001 | iadademstat |
| 4-[2-(4-aminopiperidin-1-yl)-5-(3-fluoro-4-methoxyphenyl)-1-methyl-6-oxo- | CC-90011 | pulrodemstat |
| 1,6-dihydropyrimidin-4-yl]-2-fluorobenzonitrile | | |
| N-[(2S)-5-{[(1R,2S)-2-(4-fluorophenyl)cyclopropyl]amino}-1-(4- | IMG-7289 | bomedemstat |
| methylpiperazin-1-yl)-1-oxopentan-2-yl]-4-(1H-1,2,3-triazol-1-yl)benzamide | | |
| (E)-N′-(1-(5-chloro-2-hydroxyphenyl)ethylidene)-3-((4-methylpiperazin-1- | SP-2577 | seclidemstat |
| yl)sulfonyl)benzohydrazide | | |
| 1-((4-(methoxymethyl)-4-(((1R,2S)-2- | | |
| phenylcyclopropylamino)methyl)piperidin-1- | | |
| yl)methyl)cyclobutanecarboxylic acid | | |
| 3-(cyanomethyl)-3-(4-{[(1R,2S)-2-phenylcyclopropyl]amino}piperidin-1- | | |
| yl)azetidine-1-sulfonamide | | |
| 5-((((1R,2S)-2-(4-(benzyloxy)phenyl)cyclopropyl)amino)methyl)-1,3,4- | ORY-2001 | vafidemstat |
| oxadiazol-2-amine | | |
| N-((trans)-2-phenylcyclopropyl)piperidin-4-amine | OG-668, also | |
| | known as | |
| | GSK-LSD1 | |
| N-[4-[(trans)-2-[(cyclopropylmethyl)amino]cyclopropyl]phenyl]-1-methyl-1H- | T-3775440 | |
| pyrazole-4-carboxamide | | |
| 1-morpholino-3-(4-(((1R,2S)-2-phenylcyclopropyl)amino)piperidin-1- | MRTX1519 | |
| yl)propan-1-one | | |
| 4-[[4-[[[(1R,2S)-2-phenylcyclopropyl]amino]methyl]-1-piperidinyl]methyl]- | GSK2879552 | |
| benzoic acid | | |
| 4-[5-[(3S)-3-aminopyrrolidine-1-carbonyl]-2-[2-fluoro-4-(2-hydroxy-2- | | |
| methyl-propyl)phenyl]phenyl]-2-fluoro-benzonitrile | | |
| 2-(6-(((1R,2S)-2-((E)-1-phenylbut-1-en-2-yl)cyclopropyl)amino)-2- | | |
| azaspiro[3.3]heptan-2-yl)ethanol | | |
| 2-(6-(((1R,2S)-2-((E)-1-phenylbut-1-en-2-yl)cyclopropyl)amino)-2- | | |
| azaspiro[3.3]heptan-2-yl)propane-1,3-diol | | |

The LSD1 inhibitor to be used in accordance with the present invention may thus be, e.g., any one of the specific compounds listed in the Table above, or a pharmaceutically acceptable salt of any one of these compounds.

In some embodiments, the LSD1 inhibitor is an LSD1 inhibitor known in the art, including, e.g., any one of the compounds disclosed in WO2010/043721, WO2010/084160, WO2010/143582, WO2011/035941, WO2011/042217, WO2011/131576, WO2011/131697, WO2012/013727, WO2012/013728, WO2012/045883, WO2012/135113, WO2013/022047, EP2743256A1, WO2013/025805, WO2013/057320, WO2013/057322, WO2014/058071, EP2907802A1, WO2014/084298, EP2927212A1, WO2014/086790, WO2014/164867, WO2014/194280, WO2014/205213, WO2015/021128, WO2015/031564, WO2015/089192, WO2015/120281, WO2015/123408, WO2015/123424, WO2015/123437, WO2015/123465, WO2015/134973, WO2015/168466, WO2015/181380, WO2015/200843, WO2016/003917, WO2016/004105, WO2016/007722, WO2016/007727, WO2016/007731, WO2016/007736, WO2016/034946, WO2016/037005, WO2016/123387, WO2016/130952, WO2016/161282, WO2016/172496, WO2016/177656, WO2017/004519, WO2017/027678, WO2017/079476, WO2017/079670, WO2017/090756, EP3381896A1, WO2017/109061, WO2017/116558, WO2017/149463, WO2017/157322, EP3431471A1, WO2017/184934, WO2017/195216, WO2017/198780, WO2017/215464, EP3486244A1, WO2018/081342, WO2018/081343, WO2018/137644, EP3575285A1, WO2018/213211, WO2018/216800, EP3632897A1, WO2018/226053, WO2018/234978, WO2019/009412, WO2019/034774, WO2019/054766, WO2019/217972, WO2019/222069, WO2020/015745, EP3825309A1, WO2020/047198, WO2020/052647, WO2020/052649, EP3851440A1, WO2020/138398, WO2020/159285, EP3907225A1, WO2021/058024, WO2021/095835, WO2021/175079, US2017-0283397, US2022-0064126, CN103054869, CN103319466, CN104119280, CN105541806, CN105924362, CN105985265, CN106045862, CN106045881, CN106432248, CN106478639, CN106831489, CN106928235, CN107033148 CN107174584, CN107176927, CN107459476, CN107474011, CN107501169, CN107936022, CN108530302, CN109265462, CN109293664, CN109535019, CN110204551, CN110478352, CN111072610, CN111454252, CN112110936, CN112409310, CN112920130, CN113087712, CN113105479, CN113264903, CN113582906, CN113599380, KR20190040763, or KR20190040783, each of which is incorporated herein by reference in its entirety (including, in particular, the compounds described in the examples section of each one of these documents). Accordingly, the LSD1 inhibitor may be, e.g., a compound disclosed in any one of the aforementioned documents (including, e.g., in the examples section of any one of these documents), wherein said compound may be used in non-salt form or in the form of a pharmaceutically acceptable salt.

In some embodiments, the LSD1 inhibitor is a compound selected from the group consisting of iadademstat, pulrodemstat (CC-90011), bomedemstat, seclidemstat, 1-((4-(methoxymethyl)-4-(((1R,2S)-2-phenylcyclopropylamino)methyl)piperidin-1-yl)methyl)cyclobutanecarboxylic acid, 3-(cyanomethyl)-3-(4 {[(1R,2S)-2-phenylcyclopropyl]amino}piperidin-1-yl)azetidine-1-sulfonamide, and pharmaceutically acceptable salts thereof. Iadademstat is a selective and irreversible LSD1 inhibitor. Iadademstat is the INN for the compound of formula:

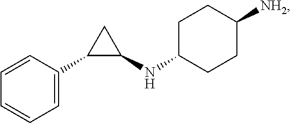 [SMILES: N[C@H]1CC[C@H](N[C@@H]2C[C@H]2c2ccccc2)CC1]

[CAS Reg. No. 1431304-21-0], which is also known as ORY-1001 or (trans)-N1-((1R,2S)-2-phenylcyclopropyl)cyclohexane-1,4-diamine. Iadademstat has been described e.g. in Example 5 of WO2013/057322. Pharmaceutically acceptable salts thereof are also described therein, including hydrochloride salts.

Pulrodemstat is a reversible LSD1 inhibitor of formula

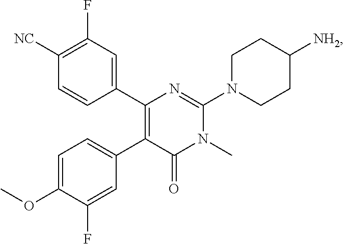 [SMILES: COc1ccc(-c2c(-c3ccc(C#N)c(F)c3)nc(N3CCC(N)CC3)n(C)c2=O)cc1F]

[CAS Reg. No. 1821307-10-1], also known as CC-90011, with chemical name 4-[2-(4-aminopiperidin-1-yl)-5-(3-fluoro-4-methoxyphenyl)-1-ethyl-6-oxo-1,6-dihydropyrimidin-4-yl]-2-fluorobenzonitrile. Pulrodemstat (CC-90011) has been described e.g. in WO2015/168466 and WO2017/79670. Pharmaceutically acceptable salts thereof are also described therein, including a besylate salt.

Bomedemstat is an irreversible LSD1 inhibitor of formula

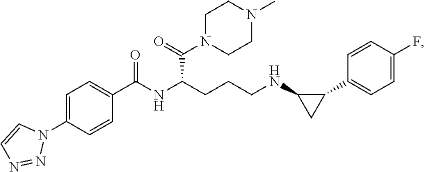 [SMILES: CN1CCN(C(=O)[C@H](CCCN[C@@H]2C[C@H]2c2ccc(F)cc2)NC(=O)c2ccc(-n3ccnn3)cc2)CC1]

[CAS Reg. No. 1990504-34-1], also known as IMG-7289, and with chemical name N-[(2S)-5-{[(1R,2S)-2-(4-fluorophenyl)cyclopropyl]amino}-1-(4-methylpiperazin-1-yl)-1-oxopentan-2-yl]-4-(1H-1,2,3-triazol-1-yl) benzamide. Bomedemstat has been described e.g. in WO2016/130952 and WO2018/35259. Pharmaceutically acceptable salts thereof are also described therein, including a bis-tosylate salt.

Seclidemstat is an LSD1 inhibitor of formula

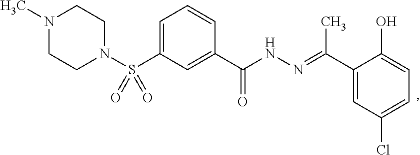 [SMILES: C/C(=N\NC(=O)c1cccc(S(=O)(=O)N2CCN(C)CC2)c1)c1cc(Cl)ccc1O]

[CAS Reg. No. 1423715-37-0], also known as SP-2577, and with chemical name (E)-N′-(1-(5-chloro-2-hydroxyphenyl)ethylidene)-3-((4-methylpiperazin-1-yl)sulfonyl)benzohydrazide. Seclidemstat has been described e.g. in WO2013/025805 and WO2014/205213.

1-((4-(Methoxymethyl)-4-(((1R,2S)-2-phenylcyclopropylamino)methyl)piperidin-1-yl)methyl)cyclobutanecarboxylic acid is an irreversible LSD1 inhibitor described e.g. in WO2015/123465 and WO2017/27678. Pharmaceutically acceptable salts thereof are also described therein, including a p-toluenesulfonate salt.

3-(Cyanomethyl)-3-(4-{[(1R,2S)-2-phenylcyclopropyl]amino}piperidin-1-yl)azetidine-1-sulfonamide is an irreversible LSD1 inhibitor described e.g. in WO2020/047198. Pharmaceutically acceptable salts thereof are also described therein. Vafidemstat is an irreversible LSD1 inhibitor of formula:

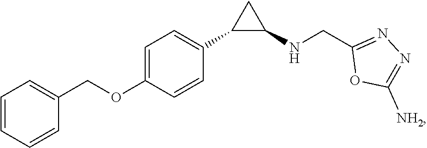 [SMILES: Nc1nnc(CN[C@@H]2C[C@H]2c2ccc(OCc3ccccc3)cc2)o1]

which is also known as ORY-2001, 5-((((1R,2S)-2-(4-(benzyloxy)phenyl)cyclopropyl)amino)methyl)-1,3,4-oxadiazol-2-amine or (-) 5-((((trans)-2-(4-(benzyloxy)phenyl)cyclopropyl)amino)methyl)-1,3,4-oxadiazol-2-amine. Vafidemstat has been described e.g. in Example 35 of WO2012/13728.

In some embodiments, the LSD1 inhibitor is selected from the group consisting of iadademstat, pulrodemstat (CC-90011), bomedemstat, and pharmaceutically acceptable salts thereof.

A particularly preferred LSD1 inhibitor is iadademstat or a pharmaceutically acceptable salt thereof. In some embodiments, iadademstat is used as the dihydrochloride salt.

### Gilteritinib

Gilteritinib is the INN for the compound of formula:

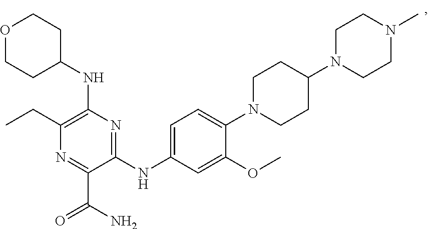 [SMILES: CCc1nc(C(N)=O)c(Nc2ccc(N3CCC(N4CCN(C)CC4)CC3)c(OC)c2)nc1NC1CCOCC1]

[CAS Reg. No. 1254053-43-4], which is also known as ASP2215 or 6-ethyl-3-[[3-methoxy-4-[4-(4-methylpiperazinyl)piperidin-1-yl]phenyl]amino]-5-[(tetrahydro-2H-pyran-4-yl)amino]pyrazine-2-carboxamide.

Gilteritinib is a FLT3 inhibitor, in particular a type | FLT3 inhibitor, and a corresponding medicinal product is sold under the tradename Xospata®. Gilteritinib is preferably used as the fumarate salt.

Unless specifically indicated otherwise, any reference to an LSD1 inhibitor (for example iadademstat) throughout the present description and claims includes such LSD1 inhibitor in non-salt form and any of its pharmaceutically acceptable salts. When the LSD1 inhibitor is iadademstat, it is preferably used in the form of a pharmaceutically acceptable salt, preferably a hydrochloride salt, more preferably the di-hydrochloride salt.

Likewise, any reference to gilteritinib throughout the present description and claims includes gilteritinib (in non-salt form) and any of its pharmaceutically acceptable salts. Preferably, gilteritinib is used in the form of a pharmaceutically acceptable salt, preferably the fumarate salt.

Administration of the combination of the LSD1 inhibitor and gilteritinib can include administering compositions in any useful format. For example, the combination of the invention may be administered using separate pharmaceutical formulations for each active ingredient (i.e. separate formulations for the LSD1 inhibitor and for gilteritinib), or may be administered using a pharmaceutical formulation comprising both the LSD1 inhibitor and gilteritinib. When using separate formulations, e.g. a first formulation comprising an LSD1 inhibitor and a second formulation comprising gilteritinib, the formulations can be administered in any order, whether sequentially or simultaneously, wherein preferably there is a time period while both (or all) active agents simultaneously exert their biological activities.

In some embodiments, one or more additional therapeutic agents can be administered to the patient. The additional therapeutic agent(s) can comprise one or more additional anticancer agents, including any agents used for the treatment of myeloid cancers, particularly AML, including any of the corresponding agents listed in the FDA's Orange Book or other reference works listing approved drugs in other countries. The additional therapeutic agent(s) may also comprise one or more antiemetic agents, such as, e.g., a 5-HT₃ antagonist (e.g., palonosetron, ramosetron, alosetron, ondansetron, tropisetron, granisetron, or dolasetron), olanzapine, a corticosteroid (e.g., methylprednisolone or dexamethasone), or prochlorperazine.

### Pharmaceutical Formulations

The LSD1 inhibitor and gilteritinib for use in the combinations as described herein as well as pharmaceutical compositions as described herein comprising a combination of the invention may be administered by any route appropriate to the condition to be treated. Suitable routes include oral, parenteral (including subcutaneous, intramuscular, intravenous, intraarterial, inhalation, intradermal, intrathecal, epidural, and infusion techniques), transdermal, rectal, nasal, topical (including buccal and sublingual), vaginal, intraperitoneal, intrapulmonary and intranasal. Preferably, both components of the combination (LSD1 inhibitor and gilteritinib) when formulated separately, or the combination when both active ingredients are formulated in a single formulation, are administered orally.

The LSD1 inhibitor and gilteritinib for use in the combinations as described herein as well as pharmaceutical compositions as described herein comprising a combination of the invention may be administered in any convenient pharmaceutical composition or formulation, e.g., as tablets, powders, capsules, solutions, dispersions, suspensions, syrups, sprays, suppositories, gels, emulsions, patches, etc. Such compositions/formulations may comprise components conventional in pharmaceutical preparations, e.g., diluents, carriers, pH modifiers, preservatives, solubilizers, stabilizers, wetting agents, emulsifiers, sweeteners, colorants, flavorants, salts for varying the osmotic pressure, buffers, masking agents, antioxidants, and/or further active agents. They can also comprise still other therapeutically active or therapeutically valuable substances.

A typical formulation is prepared by mixing an LSD1 inhibitor or gilteritinib or a combination as described herein and one or more pharmaceutically acceptable excipients. Suitable excipients are well known to those skilled in the art and are described in detail in, e.g., “Ansel's Pharmaceutical Dosage Forms and Drug Delivery Systems” (2004) Lippincott, Williams & Wilkins, Philadelphia; “Remington: The Science and Practice of Pharmacy” (2000) Lippincott, Williams & Wilkins, Philadelphia; and “Handbook of Pharmaceutical Excipients” (2005) Pharmaceutical Press, Chicago. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and/or other known additives to provide an elegant presentation of the drug (i.e., a compound of the present invention or pharmaceutical composition thereof) or aid in the manufacturing of the pharmaceutical product (i.e., medicament).

For oral delivery, the compound can be incorporated into a formulation that includes pharmaceutically acceptable carriers such as binders (e.g., gelatin, cellulose, gum tragacanth), excipients (e.g., starch, lactose), lubricants (e.g., magnesium stearate, silicon dioxide), disintegrating agents (e.g., alginate, Primogel, and corn starch), and sweetening or flavoring agents (e.g., glucose, sucrose, saccharin, methyl salicylate, and peppermint). The formulation can be orally delivered, e.g., in the form of enclosed gelatin capsules or compressed tablets. Capsules and tablets can be prepared by any conventional techniques. The capsules and tablets can also be coated with various coatings known in the art to modify the flavors, tastes, colors, and shapes of the capsules and tablets. In addition, liquid carriers such as fatty oil can also be included in capsules.

Suitable oral formulations can also be in the form of a suspension, syrup, chewing gum, wafer, elixir, and the like. If desired, conventional agents for modifying flavors, tastes, colors, and shapes of the special forms can also be included. In addition, for convenient administration by enteral feeding tube in patients unable to swallow, the active compounds can be dissolved in an acceptable lipophilic vegetable oil vehicle such as olive oil, corn oil and safflower oil.

The compound can also be administered parenterally in the form of solution or suspension, or in lyophilized form capable of conversion into a solution or suspension form before use. In such formulations, diluents or pharmaceutically acceptable carriers such as sterile water and physiological saline buffer can be used. Other conventional solvents, pH buffers, stabilizers, anti-bacterial agents, surfactants, and antioxidants can all be included. For example, useful components include sodium chloride, acetates, citrate or phosphate buffers, glycerin, dextrose, fixed oils, methyl parabens, polyethylene glycol, propylene glycol, sodium bisulfate, benzyl alcohol, ascorbic acid, and the like. The parenteral formulations can be stored in any conventional containers such as vials and ampoules.

Subcutaneous implantation for sustained release of the compound may also be a suitable route of administration. This entails surgical procedures for implanting an active compound in any suitable formulation into a subcutaneous space, e.g., beneath the anterior abdominal wall. See, e.g., Wilson et al. (1984) J. Clin. Psych. 45:242-247. Hydrogels can be used as a carrier for the sustained release of active compounds. Hydrogels are generally known in the art. They are typically made by crosslinking high molecular weight biocompatible polymers into a network, which swells in water to form a gel like material. Preferably, hydrogels are biodegradable or biosorbable. For purposes of this invention, hydrogels made of polyethylene glycols, collagen, or poly (glycolic-co-L-lactic acid) may be useful. See, e.g., Phillips et al. (1984) J. Pharmaceut. Sci., 73:1718-1720.

The pharmaceutical compositions, like oral and parenteral compositions, can be formulated in unit dosage forms for ease of administration and uniformity of dosage. As used herein, “unit dosage forms” refers to physically discrete units suitable as unitary dosages for administration to subjects, each unit containing a predetermined quantity of active ingredient calculated to produce the desired therapeutic effect, in association with one or more suitable pharmaceutical carriers.

Suitable oral dosage forms for iadademstat are disclosed, for example, in WO2019/211491A1.

In particular, iadademstat may be provided in the form of tablets. Alternatively, iadademstat may also be provided in the form of an oral aqueous solution (which may be prepared, e.g., from a powder for reconstitution). As explained above, it is preferred that iadademstat is used in the form of iadademstat dihydrochloride.

Suitable oral dosage forms for gilteritinib that can be used in the present invention include, for example, those marketed under the name Xospata®. Gilteritinib is marketed as film-coated tablets containing 40 mg of gilteritinib (as fumarate). Such tablets for oral use can be prepared using mannitol (E421), hydroxypropylcellulose, hydroxypropylcellulose (low substituted) and magnesium stearate as excipients for the tablet core, and hypromellose, talc, macrogol, titanium dioxide and iron oxide yellow (E172) as excipients for the film coating, e.g., as described in the Summary of Product Characteristics for Xospata®, which is incorporated herein by reference in its entirety (particularly in its most recent version available on Apr. 1, 2021). Accordingly, in some embodiments, gilteritinib is provided in the form of tablets (e.g., tablets containing 40 mg of gilteritinib, preferably as a fumarate salt).

In therapeutic applications, combinations and pharmaceutical compositions of the invention are to be administered in a manner appropriate to the disease to be treated, as determined by a person skilled in the medical arts. An appropriate dose and suitable duration and frequency of administration can vary within wide limits and will be determined by such factors as the condition of the patient, the type and severity of the disease, the particular form of the active ingredient(s), the method of administration, among others. In general, an appropriate dose and administration regimen provides the active ingredients of the combination of the invention in an amount sufficient to provide therapeutic benefit, for example an improved clinical outcome, such as more frequent complete or partial remissions, or longer disease-free and/or overall survival, or lessening of symptoms severity, or any other objectively identifiable improvement as noted by the clinician. Therapeutically effective doses may generally be assessed or extrapolated using experimental models like dose-response curves derived from in vitro or animal model test systems, or from clinical trials in humans.

As an example, suitable doses for gilteritinib may be those presently used in clinical practice in the treatment of AML. The current recommended dose for gilteritinib as monotherapy for the treatment of R/R AML is 120 mg daily, which can be increased to 200 mg daily. Accordingly, gilteritinib may be orally administered, e.g., in a dose of about 120 mg daily. Other doses may also be possible, for example the dose of gilteritinib may be lowered due to the combined action (synergy) of the newly identified combinations of gilteritinib with LSD1 inhibitors. Doses as reflected herein for gilteritinib relate to the corresponding amount of the gilteritinib free base.

Suitable doses and dosing regimens for the LSD1 inhibitor will be dependent on the specific LSD1 inhibitor used, its LSD1 inhibitory potency, its pharmacokinetic profile and other factors, as well known by those skilled in the art.

Iadademstat is a highly potent active pharmaceutical ingredient (HPAPI). The anticipated daily dose is thus very low, e.g., lower than 1 mg per day. Accordingly, the drug load in a solid form will also be very low, e.g., less than 1 mg of API per 100 mg of tablet. In general, in the case of oral administration (e.g., as tablets or as an oral aqueous solution), a daily dosage of about 50 ug to about 300 ug, preferably of about 75 ug to about 300 ug (e.g., about 75 ug, about 100 ug, about 125 ug, about 150 ug, about 175 ug, about 200 ug, about 225 ug, about 250 ug, about 275 ug, or about 300 ug, or any range between any two of the aforementioned daily dosages), of iadademstat as described herein should be appropriate, although these limits may be adjusted when necessary. The term “ug”, as used herein, refers to micrograms and is used synonymously with the term “μg”.

In some embodiments, the LSD1 inhibitor is iadademstat (or a pharmaceutically acceptable salt thereof, e.g., iadademstat dihydrochloride) and is administered five days on/two days off (5/2) per week.

In some embodiments, the LSD1 inhibitor is iadademstat (or a pharmaceutically acceptable salt thereof, e.g., iadademstat dihydrochloride) and is administered orally at a daily dose of about 50 ug to about 300 ug, preferably of about 75 ug to about 300 ug (e.g., about 100 ug to about 300 ug), five days on/two days off (5/2) per week. Doses as reflected herein for jadademstat relate to the corresponding amount of the iadademstat free base. In some embodiments, iadademstat is administered orally at a daily dose of about 75 ug five days on/two days off (5/2) per week. In some embodiments, iadademstat is administered orally at a daily dose of about 100 ug five days on/two days off (5/2) per week. In some embodiments, iadademstat is administered orally at a daily dose of about 150 ug five days on/two days off (5/2) per week. In some embodiments, iadademstat is administered orally at a daily dose of about 200 ug five days on/two days off (5/2) per week. In some embodiments, iadademstat is administered orally at a daily dose of about 250 ug five days on/two days off (5/2) per week. In some embodiments, iadademstat is administered orally at a daily dose of about 300 ug five days on/two days off (5/2) per week.

### Articles of Manufacture

The combinations and pharmaceutical compositions of the invention can be included in a container, pack or dispenser together with instructions for administration.

In another embodiment of the invention, an article of manufacture, or “kit”, containing a combination as described herein is provided.

In some embodiments, the article of manufacture or kit comprises a container and a combination according to the invention as described herein.

In some embodiments, the article of manufacture or kit comprises: a) a container comprising the LSD1 inhibitor (or a pharmaceutically acceptable salt thereof), and b) a container comprising gilteritinib (or a pharmaceutically acceptable salt thereof).

The articles of manufacture or kits may further comprise a label or package insert. The term “package insert” is used to refer to instructions customarily included in commercial packages of therapeutic products, that contain information about the indications, usage, dosage, administration, contraindications and/or warnings concerning the use of such therapeutic products. Suitable containers include, for example, blister packs, bottles, vials, syringes, etc. The container may be formed from a variety of materials such as glass or plastic. The container may hold a combination, or a formulation thereof, which is effective for treating the condition and may have a sterile access port (for example, the container may be an intravenous solution bag or a vial having a stopper pierceable by a hypodermic injection needle). The label or package insert indicates that the composition is used for treating the condition of choice, such as AML. Alternatively, or additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kit may further comprise directions for the administration of the combination, and, if present, the second pharmaceutical formulation. For example, if the kit comprises a first pharmaceutical composition/formulation comprising the LSD1 inhibitor or a pharmaceutically acceptable salt thereof and a second pharmaceutical composition/formulation comprising gilteritinib or a pharmaceutically acceptable salt thereof, the kit may further comprise directions for the simultaneous, sequential or separate administration of the first and second pharmaceutical compositions/formulations to a patient in need thereof.

In another embodiment, the kit is suitable for the delivery of solid oral forms of a combination, such as tablets or capsules. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. If desired, a memory aid can be provided, for example in the form of numbers, letters, or other markings or with a calendar insert, designating the days in the treatment schedule in which the dosages can be administered.

According to one embodiment, a kit may comprise (a) a first container with the LSD1 inhibitor or a pharmaceutically acceptable salt thereof contained therein; (b) a second container with gilteritinib or a pharmaceutically acceptable salt thereof; and (c) a third container with a third pharmaceutical formulation contained therein, wherein the third pharmaceutical formulation comprises another compound with anticancer activity. Alternatively, or additionally, the kit may comprise another container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Where the kit comprises a composition of the LSD1 inhibitor or a pharmaceutically acceptable salt thereof and a composition of gilteritinib or a pharmaceutically acceptable salt thereof, the kit may comprise a container for containing the separate compositions such as a divided bottle or a divided foil packet, however, the separate compositions may also be contained within a single, undivided container. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

### Definitions Unless defined otherwise, all technical and scientific terms used herein have the same meaning as commonly understood by one of ordinary skill in the art to which this invention pertains.

The following definitions apply throughout the present specification and claims, unless specifically indicated otherwise. A “patient” or “subject” for the purposes of the present invention includes both humans and other animals, particularly mammals. Thus, the methods and uses of the invention are applicable to both human therapy and veterinary applications. In a preferred aspect the subject or patient is a mammal, and in the most preferred aspect the subject or patient is a human (e.g. a male or female human).

The terms “treatment”, “treating” and the like are used herein to generally mean obtaining a desired pharmacological and/or physiological effect. This includes partially or completely curing or ameliorating a disease (i.e. cancer) and/or a symptom or adverse effect attributed to the disease or partially or completely halting the progression of a disease and/or a symptom or adverse effect attributed to the disease. The term “treatment” as used herein covers any treatment of a disease (i.e. cancer) in a patient and includes, without limitation, inhibiting cancer, i.e. arresting, delaying or slowing down its development/progression; or relieving the cancer, i.e. causing (complete or partial) regression, correction or alleviation of cancer. The present invention specifically and distinctly relates to each one of these forms of treatment.

As used herein, the term “therapeutically effective amount” or “effective amount” of a compound or combination according to the invention refers to an amount sufficient to produce a desired biological effect (e.g., a therapeutic effect or benefit) in a subject. Accordingly, a therapeutically effective amount of a compound or combination may be an amount which is sufficient to treat a disease (i.e. cancer), and/or delay the onset or progression of the disease, and/or alleviate one or more symptoms of the disease, when administered to a subject suffering from or susceptible to that disease. The therapeutically effective amount will vary depending on the compound, the disease state being treated, the severity of the disease treated, the age and relative health of the subject, the route and form of administration, the judgment of the attending medical or veterinary practitioner, and other factors.

The term “pharmaceutically acceptable” denotes an attribute of a material which is useful in preparing a pharmaceutical composition that is generally safe, non-toxic, and neither biologically nor otherwise undesirable and is acceptable for veterinary and/or human pharmaceutical use.

As used herein, a “pharmaceutically acceptable salt” is intended to mean a salt that retains the biological effectiveness of the free acids and/or bases of the specified compound and that is not biologically or otherwise undesirable. A compound may possess a sufficiently acidic, a sufficiently basic, or both functional groups, and accordingly react with any of a number of inorganic or organic bases, and inorganic or organic acids, to form a pharmaceutically acceptable salt. Exemplary pharmaceutically acceptable salts include those salts prepared by reaction of a compound according to the invention, e.g. iadademstat, with a mineral or organic acid, such as hydrochlorides, hydrobromides, sulfates, pyrosulfates, bisulfates, sulfites, bisulfites, phosphates, monohydrophosphates, dihydrophosphates, metaphosphates, pyrophosphates, chlorides, bromides, iodides, nitrates, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, gamma-hydroxybutyrates, glycollates, tartrates, methane-sulfonates (or mesylates), ethane-sulfonates, propanesulfonates, benzenesulfonates (or besylates), toluenesulfonates, trifluoromethansulfonates, naphthalene-1-sulfonates, naphthalene-2-sulfonates, mandelates, pyruvates, stearates, ascorbates, or salicylates. When a compound carries an acidic moiety, suitable pharmaceutically acceptable salts thereof may include alkali metal salts, e.g. sodium or potassium salts; alkaline earth metal salts, e.g. calcium or magnesium salts; and salts formed with suitable organic ligands such as ammonia, alkylamines, hydroxyalkylamines, lysine, arginine, N-methylglucamine, procaine and the like. Pharmaceutically acceptable salts are well known in the art.

The terms “pharmaceutical composition” and “pharmaceutical formulation” (or “formulation”) are used interchangeably and denote a mixture or solution comprising a therapeutically effective amount of an active pharmaceutical ingredient or combination of the invention together with one or more pharmaceutically acceptable excipients to be administered to a mammal, e.g., a human in need thereof.

The terms “pharmaceutically acceptable excipient” or “pharmaceutically acceptable carrier” can be used interchangeably and denote any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents, lubricants and the like used in formulating pharmaceutical products. They are generally safe for administering to humans according to established governmental standards, including those promulgated by the United States Food and Drug Administration and/or the European Medicines Agency. Pharmaceutically acceptable carriers or excipients are well known to those skilled in the art.

The term “inhibitor” as used herein denotes a compound which competes with, decreases, blocks, inhibits, abrogates or interferes in any way with the binding of a particular ligand to a particular receptor or enzyme and/or which decreases, blocks, inhibits, abrogates or interferes in any way with the activity or function of a particular protein, e.g. of a receptor or enzyme.

As used herein, a “small molecule” refers to an organic compound with a molecular weight equal to or below 900 daltons, preferably below 500 daltons. The molecular weight is the mass of a molecule and is calculated as the sum of the atomic weights of each constituent element multiplied by the number of atoms of that element in the molecular formula.

As used herein, the term “comprising” (or “comprise”, “comprises”, “contain”, “contains”, or “containing”), unless explicitly indicated otherwise or contradicted by context, has the meaning of “containing, inter alia”, i.e., “containing, among further optional elements, . . . ”. In addition thereto, this term also includes the narrower meanings of “consisting essentially of” and “consisting of”. For example, the term “A comprising B and C” has the meaning of “A containing, inter alia, B and C”, wherein A may contain further optional elements (e.g., “A containing B, C and D” would also be encompassed), but this term also includes the meaning of “A consisting essentially of B and C” and the meaning of “A consisting of B and C” (i.e., no other components than B and C are comprised in A).

As used herein, the indefinite articles “a” and “an” and the definite article “the” include plural as well as singular referents, unless the context clearly dictates otherwise.

The term “about” or “approximately” means an acceptable error for a particular value as determined by one of ordinary skilled in the art, which depends in part on how the value is measured or determined. In certain embodiments, the term “about” or “approximately” means within 1, 2, 3 or 4 standard deviations. In certain embodiments, the term “about” or “approximately” means within 25%, 20%, 15%, 10%, 9%, 8%, 7%, 6%, 5%, 4%, 3%, 2%, 1%, 0.5%, 0.1% or 0.05% of a given value or range. Any reference to a numerical value or range provided in connection with the term “about” also includes a reference to the corresponding specific value or range.

All publications, patent applications, patents, and other references mentioned herein are incorporated by reference in their entirety.

### EXAMPLES

The following examples are provided for illustration of the invention. They should not be considered as limiting the scope of the invention, but merely as being representative thereof.

### Example 1—Matrix Assay for Determination of Synergism Between LSD1 Inhibitors and Gilteritinib in AML Cell Lines

The objective of this assay is to determine synergism existing between LSD1 inhibitors and gilteritinib. As a first step, the compounds of interest were evaluated as single agents, prior to setting up the matrix experiments to determine synergy.

### 1.1 Experimental Design

### 1.1.1 Cell Lines and Culture Conditions

Mycoplasma-free AML cell lines were maintained in RPMI 10% FBS medium at 37° C. in a humidified incubator with controlled 5% CO₂ atmosphere. Cell freezing and thawing was performed following recommendation from ATCC. Genetic profiling of the cell lines used is available in Table 1.

TABLE 1

| Cell lines | Tissue of origin | Mutational status |
|---|---|---|
| MV(4;11) | Acute monocytic leukemia (M5b) | MLL-AF4 rearranged, FLT3-ITD |
| OCI-AML3 | Acute myelomonocytic leukemia (M4) | MLL wt, NPM1 mut, DNMT3a mut |
| MOLM-13 | Acute monoblastic leukemia (M5a) | MLL-AF9, FLT3-ITD |
| TF1a | Acute erythroid leukemia (M6) | CBFA2T3-ABHD12 fusion |

### 1.1.2 Single Agent Viability Assays (96 Hours)

Cells were seeded in 96-well plates at the optimal density to guarantee linear growth throughout the assay (8000 cells/well for MV(4; 11), 4000 cells/well for MOLM-13 and OCI-AML3, 2000 cells/well for TF1a) in 50 μL of medium. Each experimental condition was tested in technical triplicates, including medium-only and vehicle-treated controls for background correction and normalization, respectively. After seeding, 50 μL of medium containing 9 serial dilutions (1:3) of 2×-concentrated compound were added to the cells to obtain 100 μL of cells with 1×-concentrated compound at each dilution. Cells were then incubated for 96 hours at 37° C. in a controlled 5% CO₂ atmosphere, prior to evaluation of cell viability using alamarBlue™ cell viability reagent (ThermoFisher Scientific, Waltham, MA/USA). AlamarBlue™ is a cell viability indicator that uses the natural reducing power of living cells to convert resazurin to the fluorescent molecule, resorufin. Briefly, alamarBlue™ stock solution was diluted 1:20 in the culture medium and after 3 hours incubation fluorescence was measured using a TECAN Infinity 2000 plate reader (Tecan Group Ltd., Männedorf, CH; 540-570 nm excitation wavelength, 580-610 nm emission wavelength). For each condition, the average fluorescence was calculated from the 3 technical replicates; background correction was calculated from the fluorescence of medium-only controls. Data were analyzed using the GraphPad PRISM® version 9.0.1 (GraphPad Software, Inc., La Jolla, CA/USA) to calculate the best-fitting curves and the EC₅₀ values.

### 1.1.3 9×9 Matrix Viability Assays (96 Hours)

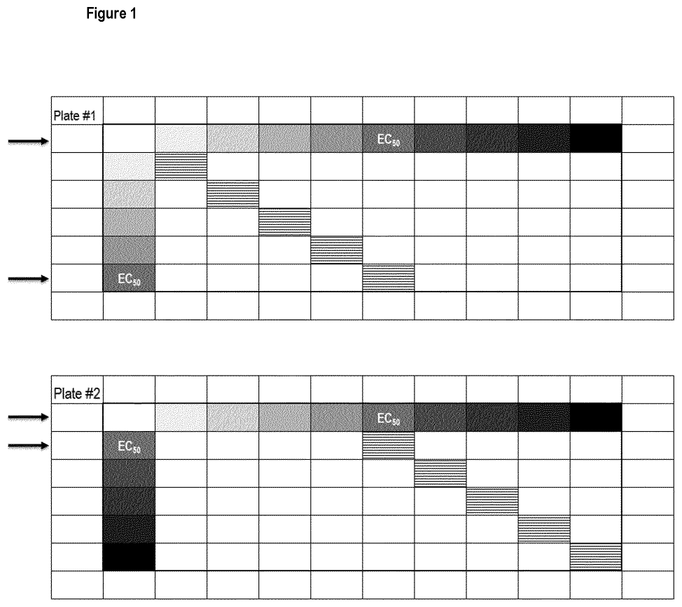

Each matrix assay was distributed across 2 plates following the scheme illustrated in FIG. 1, where one compound is added at increasing concentrations from left to right, and the other compound is added at increasing concentrations from top to bottom.

For the assay, cells were seeded in 96-well plates at the optimal density specified in the previous section in 50 μL of medium; the wells at the edges of the plates were left with 100 μL of medium-only for background correction. Each of the two compounds was added at a 4×-concentration in 25 μL, resulting in a final volume of 100 μL and final concentration of 1× at each dilution. As shown in FIG. 1, the matrix was designed with increasing concentrations of LSD1 inhibitor from left to right and increasing concentrations of gilteritinib from top to the bottom. The first and the last row of plate #1 have been repeated in plate #2 (indicated by arrows in FIG. 1), to confirm reproducibility across the two plates. The concentrations tested for both compounds covered a 6561-fold range obtained through a total of 9× 1:3 dilutions, designed to have the EC₅₀s of both compounds centered horizontally and vertically on the matrix (the EC₅₀s of the LSD1 inhibitor and gilteritinib correspond to the 5th well from the right and from the bottom respectively, as indicated in FIG. 1). In this way, the wells on the diagonal of the plates (marked with horizontal lines in FIG. 1) correspond to the fixed EC₅₀ ratios between both compounds. The EC₅₀ values for the compounds tested in the matrix assays were previously obtained through single agent assays performed as detailed in section 1.1.2.

Viability was then determined using alamarBlue™ staining as detailed in section 1.1.2. in at least two independent biological replicates (N=2).

### 1.1.3.1 9×9 Matrix Viability Assays (Data Analysis)

For each matrix assay, data were then normalized against the vehicle-treated controls (≤0.5% DMSO, in the upper left corner) to obtain the percentage value of relative residual viability, according to the following formula:

% relative residual viability=Background-corrected RFU treated cells/Background-corrected RFU vehicle control×100

The values for percentage of residual viability were then analyzed using GraphPad PRISM® version 9.0.1 (GraphPad Software, Inc., La Jolla, CA/USA) to calculate the best-fitting curve and the EC₅₀ values on the single agents.

At this point the Fraction affected (Fa), also known as Fractional Effect, was calculated using the formula:

Fa=1−(% relative residual viability/100)

for the following conditions:
Cells treated with serial dilutions of LSD1 inhibitor as single agent (average of the first row of the first and second plate of each matrix assay)
Cells treated with serial dilutions of gilteritinib as single agent (in the first column of the matrix assay)
Cells treated with LSD1 inhibitors and gilteritinib at a fixed ratio corresponding to the ratio of EC₅₀ values (values of % relative residual viability in the diagonal of the matrix assay; highlighted in FIG. 1).

The CalcuSyn software (http://www.biosoft.com/w/calcusyn.htm, Biosoft, Cambridge, UK) is designed to determine the nature (synergistic, additive or antagonistic) of the interaction between two compounds by calculating a Combination Index (CI). This analysis is based on the Median Effect Principle and the Combination Index Theorem described by the Chou-Talalay method (T. C. Chou, Pharmacol Rev. 2006; 58(3):621-681), where a resulting CI<1 is indicative of synergistic effects, a CI=1 indicates additive affects, while CI>1 reflects antagonistic effects. In the case of synergistic effects (CI<1), the smaller the CI value is, the stronger the synergy. Additionally, the strength of the drug interactions can be further classified based on the CI range, as shown in Table 2.

TABLE 2

| | Cl | Classification |
|---|---|---|
| [symbol] | <0.1 0.1-0.3 0.3-0.7 0.7-0.85 0.85-0.9 | +++++ ++++ +++ ++ + |
| Additive | 0.9-1.1 | +/− |
| [symbol] | 1.1-1.20 1.20-1.45 1.45-3.3 3.3-10 >10 | − −− −−− −−−− −−−−− |

In order to generate informative and consistent results, the data processed with Calcusyn (both for the single agents and the drug combination) need to fit with the Median Effect Principle and the Combination Index Theorem theoretical models. For this reason, it is crucial to remove possible outliers and data points characterized by poor fit to the Median Effect Principle (T. C. Chou, Pharmacol Rev. 2006; 58(3):621-681). In order to achieve this, the following strategy was adopted for data filtering:

In the first step data dispersion was reduced removing points characterized by:
1) Fa<0.1
2) Increase in Fa<0.03, compared to the previous point (if Fa>0.9).

These conditions define the plateaus of the dose response curve, in which cells have been treated with very low or very high concentrations of compounds (or combos), resulting in reduction of viability close to 0% or 100% (equivalent to Fa value close to 0 or 1, respectively). To be noted, in these areas of the dose-response curves the changes in alamarBlue™ signal are very small and most likely due to random noise with very little biological significance.

Next, for each data point, Log₁₀ (Concentration) and Log₁₀ (Fa/(1−Fa) were calculated and a dot plot graph was generated reporting the former value on the x axis and the latter on the y axis. With Excel, a regression line was then obtained (corresponding to the Median Effect Equation).

At this point the distance from the regression line was calculated for each data point with the equation:

$$Distance ( a x + b y + c = 0 ; X , Y ) = ( a X + b Y + c ) / √ ( {a}^{2} + {b}^{2} )$$

Outliers are identified on the basis of their distance from the Median Effect Equation, using the Grubbs's test. For each data point, the Grubbs's test was performed on the absolute value of the distance, according to the following formula (to be noted, the variable for the Grubbs's test can be called interchangeably G or Z):

$$G = ( {X}_{n} - {X}_{average} ) / s$$

Where Xₙ stands for the absolute value of the distance of each point from the regression line; X_{average} stands for average of all the Xₙ values and s stands for the standard deviation. Values of G above G_{crit} (calculated for α=0.2 as shown below) identify outliers not fitting on the Median Effect Equation. Such data points have been removed to successfully calculate the Combination Index with CalcuSyn.

$${G}_{crit} = \frac{( n - 1 ) {t}_{crit}}{\sqrt{n ( n - 2 + {t}_{crit}^{2} )}}$$

When possible, the test was reiterated more than once to remove multiple outliers, until:
1. no further outliers were identified or
2. R²>0.95. To measure data quality, the R value is calculated also by the CalcuSyn software (good data are characterized by R value above 0.95).

### 1.1.3.2 CalcuSyn Output

CalcuSyn results are provided as the experimental Fractional Effect (referred as Fa) representing the fraction of cells affected by the combined treatment at their fixed EC₅₀ ratio (in the case of a cytotoxic treatment the Fractional Effect corresponds to viability reduction compared to vehicle controls, where Fa=1 is equal to 100% viability reduction) and the associated combination index (CI). As shown in Table 2 above, the CI value is indicative of the nature and strength of the compounds' interaction, with values below 1 representing synergistic interactions (the closer the value to 0, the stronger the synergistic effects), values equal to 1 representing additive interactions and values above 1 representing antagonistic interactions.

### 1.2 Results

### 1.2.1 Single Agent Viability: Iadademstat, Pulrodemstat (CC-90011), Gilteritinib, Bomedemstat

MV(4; 11), OCI-AML3, MOLM-13 and TF1a cell lines were seeded and incubated with either vehicle (DMSO 0.05%) or serial 1:3 dilutions of iadademstat (concentration range from 0.0014 to 9 nM) as described in section 1.1.2. In all cases, iadademstat induced a reduction of viability greater than 20% (compared to vehicle controls) with EC₅₀ values in the sub-nanomolar range in at least two biological replicates. For CC-90011, MV(4; 11) and MOLM-13 cells were treated with either vehicle (DMSO 0.05%) or serial 1:3 dilutions (concentration range from 0.045 to 300 nM) as described in section 1.1.2. In all cases, CC-90011 induced a reduction of viability greater than 20% (compared to vehicle controls) with EC₅₀ values in the nanomolar range in at least two biological replicates. For gilteritinib EC₅₀ determinations, MV(4; 11), OCI-AML3, MOLM-13 and TF1a cell lines were incubated with either vehicle (DMSO 0.45%) or serial 1:3 dilutions (concentration range from 0.014 to 90 nM for MOLM-13 and MV(4; 11) and 1.4 to 9000 nM for TF1a and OCI-AML3) as described in section 1.1.2. In cell lines with FLT3-ITD, MOLM-13 and MV(4; 11), gilteritinib showed a marked viability reduction of nearly 100% in both cell lines with an EC₅₀ in the nanomolar range. In cells without FLT3 mutations such as TF1a or OCI-AML3 cells, gilteritinib induced a viability reduction >70% with an EC₅₀ in the micromolar range for both cell lines. For bomedemstat, MV(4; 11) and MOLM-13 cells were treated with either vehicle (DMSO 0.05%) or serial 1:3 dilutions (concentration range from 0.045 to 300 nM) as described in section 1.1.2. In all cases, bomedemstat induced a reduction of viability greater than 20% (compared to vehicle controls) with EC₅₀ values in the nanomolar range. Experiments were done in at least two biological replicates.

Table 3 shows the experimentally determined EC₅₀ values after 96 hours incubation with iadademstat, CC-90011, gilteritinib and bomedemstat in the specified cell lines.

TABLE 3

| | EC₅₀ (nM) | | | |
|---|---|---|---|---|
| | MOLM-13 | MV(4; 11) | OCI-AML3 | TF-1a |
| iadademstat | 0.39 | 0.43 | 0.38 | 0.35 |
| CC-90011 | 1.49 | 2.30 | Not tested | Not tested |
| gilteritinib | 2.91 | 0.77 | 1157.00 | 1023.00 |
| bomedemstat | 6.08 | 3.73 | Not tested | Not tested |

### 1.2.2 Combination of the LSD1 Inhibitor Iadademstat+Gilteritinib

Matrix treatments with gilteritinib (concentration range from 0.014 to 90 nM for MOLM-13 and MV(4; 11) and 1.4 to 9000 nM for TF1a and OCI-AML3) and the covalent and irreversible LSD1 inhibitor iadademstat (concentration range from 0.0014 to 9 nM for all four cell lines) were performed as described in section 1.1.3. Data analysis and calculation of combination indexes were performed as described in section 1.1.3.1. The results of the combination indexes (CI) associated with specific fractional effects (Fa) and the respective classifications (as described in Table 2) obtained from the combination of iadademstat and gilteritinib are shown in Table 4.

In summary, the combination iadademstat+gilteritinib showed strong synergism in a wide range of fractional effects (Fa) in gilteritinib-sensitive cell lines carrying FLT3 mutations (MOLM-13, N=3 and MV(4; 11), N=2). Importantly, strong synergism was also observed in cell lines without FLT3 mutations (WT FLT3) which are poorly responsive to gilteritinib as single agent (OCI-AML3, N=2 and TF1a, N=3). These cell lines are also resistant to other current AML therapies. In particular, OCI-AML3 and TF1a cells were resistant to venetoclax (EC₅₀>10 μM, tested following the method described above). These results open up the possibility of successfully combining LSD1 inhibitors such as iadademstat with gilteritinib in AML patients, with or without FLT3 mutations, or in refractory/relapsed situations.

TABLE 4

| | iadademstat + gilteritinib | | | | | | | | |
|---|---|---|---|---|---|---|---|---|---|
| Cell | Experiment 1 | | | Experiment 2 | | | Experiment 3 | | |
| line | Fa | CI | Classification | Fa | CI | Classification | Fa | CI | Classification |
| MOLM-13 | 0.1990 | 0.76 | ++ | 0.17062 | 0.08 | +++++ | 0.1724 | 0.62 | +++ |
| | 0.2229 | 1.39 | −− | 0.19469 | 0.20 | ++++ | 0.2579 | 0.97 | +/− |
| | 0.5115 | 0.85 | ++ | 0.25044 | 0.38 | +++ | 0.5807 | 0.59 | +++ |
| | 0.6813 | 0.78 | ++ | 0.30029 | 0.82 | ++ | 0.8633 | 0.35 | +++ |
| | 0.9840 | 0.03 | +++++ | 0.94306 | 0.30 | ++++ | 0.9559 | 0.30 | ++++ |
| | | | | 0.98860 | 0.13 | ++++ | | | |
| MV(4; 11) | 0.3092 | 0.26 | ++++ | 0.11055 | 1.21 | −− | | | |
| | 0.3140 | 0.76 | ++ | 0.13838 | 1.31 | −− | | | |
| | 0.7449 | 0.49 | +++ | 0.33238 | 0.29 | ++++ | | | |
| | 0.7995 | 0.94 | +/− | 0.45164 | 0.50 | +++ | | | |
| | 0.9553 | 0.26 | ++++ | 0.70006 | 0.56 | +++ | | | |
| | 0.9833 | 0.19 | ++++ | 0.90462 | 0.45 | +++ | | | |
| OCI-AML3 | 0.1601 | 0.48 | +++ | | | | | | |
| | 0.1537 | 1.75 | −−− | | | | 0.1010 | 3.07 | −−− |
| | 0.3427 | 0.47 | +++ | | | | 0.2128 | 1.52 | −−− |
| | 0.5942 | 0.62 | +++ | | | | 0.5285 | 0.77 | ++ |
| | 0.8423 | 0.72 | ++ | | | | 0.8053 | 0.73 | ++ |
| | 0.9546 | 0.78 | ++ | | | | 0.9285 | 0.83 | ++ |
| TF-1a | 0.3535 | 0.89 | + | 0.61136 | 0.45 | +++ | 0.5695 | 0.45 | +++ |
| | 0.8311 | 0.14 | ++++ | 0.88147 | 0.11 | ++++ | 0.8510 | 0.10 | ++++ |
| | 0.8639 | 0.30 | ++++ | 0.89317 | 0.27 | ++++ | 0.8460 | 0.33 | +++ |
| | 0.9008 | 0.55 | +++ | 0.92790 | 0.40 | +++ | 0.8991 | 0.44 | +++ |

### 1.2.3 Combination of the LSD1 Inhibitor Pulrodemstat (CC-90011)+Gilteritinib

The synergistic effects between LSD1 inhibitors and gilteritinib described in section 1.2.2 were further confirmed using another LSD1 inhibitor, in particular the structurally unrelated, reversible LSD1 inhibitor CC-90011. Matrix treatments with gilteritinib (concentration range from 0.014 to 90 nM for MOLM-13 and MV(4; 11) and CC-90011 (concentration range from 0.045 to 300 nM for both cell lines) were performed as described in section 1.1.3. Data analysis and calculation of combination indexes were performed as described in section 1.1.3.1. The results of the combination indexes (CI) associated with specific fractional effects (Fa) and the respective classifications (as described in Table 2) obtained from the combination of CC-90011 and gilteritinib are shown in Table 5.

In summary, the combination CC-90011+gilteritinib also showed strong synergism in a wide range of fractional effects (Fa) in the tested cell lines (MOLM-13, N=2 and MV(4; 11), N=2).

TABLE 5

| | CC-90011 + gilteritinib | | | | | |
|---|---|---|---|---|---|---|
| | Experiment 1 | | | Experiment 2 | | |
| Cell | | | Classifi- | | | Classifi- |
| line | Fa | CI | cation | Fa | CI | cation |
| MOLM-13 | 0.19 | 0.49 | +++ | 0.12 | 0.59 | +++ |
| | 0.39 | 0.49 | +++ | 0.22 | 0.29 | ++++ |
| | 0.78 | 0.86 | +/− | 0.27 | 0.53 | +++ |
| | 0.95 | 0.57 | +++ | 0.52 | 0.39 | +++ |
| | | | | 0.70 | 0.51 | +++ |
| | | | | 0.90 | 0.31 | +++ |
| | | | | 0.96 | 0.28 | ++++ |
| MV(4; 11) | 0.18 | 0.97 | +/− | 0.14 | 0.59 | +++ |
| | 0.17 | 3.36 | −−−− | 0.16 | 1.03 | +/− |
| | 0.44 | 0.67 | ++ | 0.41 | 0.43 | +++ |
| | 0.72 | 0.38 | +++ | 0.70 | 0.57 | +++ |
| | 0.88 | 0.27 | ++++ | 0.93 | 0.57 | +++ |
| | 0.95 | 0.21 | ++++ | 0.99 | 0.57 | +++ |

### 1.2.4 Combination of the LSD1 Inhibitor Bomedemstat+Gilteritinib

The synergistic effects between LSD1 inhibitors and gilteritinib described in sections 1.2.2 and 1.2.3 were further confirmed using another LSD1 inhibitor, bomedemstat.

Matrix treatments with gilteritinib (concentration range from 0.014 to 90 nM for MOLM-13 and MV(4; 11) and bomedemstat (concentration range from 0.045 to 300 nM for both cell lines) were performed as described in section 1.1.3. Data analysis and calculation of combination indexes were performed as described in section 1.1.3.1. The results of the combination indexes (CI) associated with specific fractional effects (Fa) and the respective classifications (as described in Table 2) obtained from the combination of bomedemstat and gilteritinib are shown in Table 6.

In summary, the combination bomedemstat+gilteritinib showed synergy in a wide range of fractional effects (Fa) in the tested cell lines (MOLM-13, N=2 and MV(4; 11), N=2).

TABLE 6

| | bomedemstat + gilteritinib | | | | | |
|---|---|---|---|---|---|---|
| | Experiment 1 | | | Experiment 2 | | |
| Cell | | | Classifi- | | | Classifi- |
| line | Fa | CI | cation | Fa | CI | cation |
| MOLM-13 | 0.14 | 0.93 | +/− | 0.18 | 0.47 | +++ |
| | 0.15 | 2.63 | −−− | 0.23 | 0.97 | +/− |
| | 0.43 | 1.32 | −− | 0.37 | 1.34 | −− |
| | 0.77 | 0.73 | ++ | 0.61 | 1.27 | −− |
| | 0.96 | 0.25 | ++++ | 0.83 | 1.00 | +/− |
| | | | | 0.97 | 0.28 | ++++ |
| MV(4; 11) | 0.26 | 0.46 | +++ | 0.13 | 0.98 | +/− |
| | 0.40 | 0.58 | +++ | 0.25 | 0.86 | + |
| | 0.50 | 1.06 | +/− | 0.44 | 0.95 | +/− |
| | 0.71 | 1.03 | +/− | 0.65 | 1.02 | +/− |
| | 0.91 | 0.53 | +++ | 0.88 | 0.66 | +++ |
| | 0.97 | 0.27 | ++++ | 0.98 | 0.22 | ++++ |

Using the methods described in Example 1, the superior therapeutic effects of combinations of other LSD1 inhibitors with gilteritinib can be verified.

Likewise, using similar methods to the ones described in this Example 1, the superior therapeutic effects of combinations of LSD1 inhibitors with gilteritinib in other myeloid malignancies like MDS can be verified.

While the invention has been described in connection with specific embodiments thereof, it will be understood that it is capable of further modifications and this patent or patent application is intended to cover any variations, uses or adaptations of the invention following, in general, the principles of the invention and including such departures from the present disclosure as come within known or customary practice within the art to which the invention pertains and as may be applied to the essential features hereinbefore set forth and as follows in the appended claims.

1. A combination product comprising, in the same pharmaceutical formulation or in separate pharmaceutical formulations, an LSD1 inhibitor or a pharmaceutically acceptable salt thereof and gilteritinib or a pharmaceutically acceptable salt thereof.

2. The combination product according to claim 1, wherein the LSD1 inhibitor is a small molecule.

3. The combination product according to claim 1 or 2, wherein the LSD1 inhibitor is selected from the group consisting of iadademstat, pulrodemstat, bomedemstat, seclidemstat, 1-((4-(methoxymethyl)-4-(((1R,2S)-2-phenylcyclopropylamino)methyl)piperidin-1-yl)methyl) cyclobutanecarboxylic acid, 3-(cyanomethyl)-3-(4-{[(1R,2S)-2-phenylcyclopropyl]amino}piperidin-1-yl)azetidine-1-sulfonamide, and pharmaceutically acceptable salts thereof.

4. The combination product according to claim 1, wherein the LSD1 inhibitor is iadademstat or a pharmaceutically acceptable salt thereof.

5. The combination product according to claim 4, wherein the LSD1 inhibitor is iadademstat dihydrochloride.

6. The combination product according to any one of claims 1 to 5, wherein the LSD1 inhibitor or the pharmaceutically acceptable salt thereof and gilteritinib or the pharmaceutically acceptable salt thereof are provided in the same pharmaceutical formulation.

7. The combination product according to any one of claims 1 to 5, wherein the LSD1 inhibitor or the pharmaceutically acceptable salt thereof and gilteritinib or the pharmaceutically acceptable salt thereof are provided in separate pharmaceutical formulations.

8. A pharmaceutical composition comprising an LSD1 inhibitor or a pharmaceutically acceptable salt thereof and gilteritinib or a pharmaceutically acceptable salt thereof, and one or more pharmaceutically acceptable excipients.

9. The pharmaceutical composition according to claim 8, wherein the LSD1 inhibitor is a small molecule.

10. The pharmaceutical composition according to claim 8 or 9, wherein the LSD1 inhibitor is selected from the group consisting of iadademstat, pulrodemstat, bomedemstat, seclidemstat, 1-((4-(methoxymethyl)-4-(((1R,2S)-2-phenylcyclopropylamino)methyl)piperidin-1-yl)methyl)cyclobutanecarboxylic acid, 3-(cyanomethyl)-3-(4-{[(1R,2S)-2-phenylcyclopropyl]amino}piperidin-1-yl)azetidine-1-sulfonamide, and pharmaceutically acceptable salts thereof.

11. The pharmaceutical composition according to claim 8, wherein the LSD1 inhibitor is iadademstat or a pharmaceutically acceptable salt thereof.

12. The pharmaceutical composition according to claim 8, wherein the LSD1 inhibitor is iadademstat dihydrochloride.

13. An article of manufacture comprising, in the same pharmaceutical formulation or in separate pharmaceutical formulations, an LSD1 inhibitor or a pharmaceutically acceptable salt thereof and gilteritinib or a pharmaceutically acceptable salt thereof.

14. The article of manufacture according to claim 13, wherein the LSD1 inhibitor is a small molecule.

15. The article of manufacture according to claim 13 or 14, wherein the LSD1 inhibitor is selected from the group consisting of iadademstat, pulrodemstat, bomedemstat, seclidemstat, 1-((4-(methoxymethyl)-4-(((1R,2S)-2-phenylcyclopropylamino)methyl)piperidin-1-yl)methyl)cyclobutanecarboxylic acid, 3-(cyanomethyl)-3-(4-{[(1R,2S)-2-phenylcyclopropyl]amino}piperidin-1-yl)azetidine-1-sulfonamide, and pharmaceutically acceptable salts thereof.

16. The article of manufacture according to claim 13, wherein the LSD1 inhibitor is iadademstat or a pharmaceutically acceptable salt thereof.

17. The article of manufacture according to claim 13, wherein the LSD1 inhibitor is iadademstat dihydrochloride.

18. A combination product according to any one of claims 1 to 7 or an article of manufacture according to any one of claims 13 to 17 for use in therapy.

19. A combination product according to any one of claims 1 to 7 or a pharmaceutical composition according to any one of claims 8 to 12 or an article of manufacture according to any one of claims 13 to 17 for use in the treatment of a myeloid cancer.

20. A compound which is an LSD1 inhibitor or a pharmaceutically acceptable salt thereof, for use in the treatment of a myeloid cancer, wherein the LSD1 inhibitor or the pharmaceutically acceptable salt thereof is for use in combination with gilteritinib or a pharmaceutically acceptable salt thereof.

21. A compound which is gilteritinib or a pharmaceutically acceptable salt thereof, for use in the treatment of a myeloid cancer, wherein gilteritinib or the pharmaceutically acceptable salt thereof is for use in combination with an LSD1 inhibitor or a pharmaceutically acceptable salt thereof.

22. The compound for use according to claim 20 or 21, wherein the LSD1 inhibitor is a small molecule.

23. The compound for use according to claim 20 or 21, wherein the LSD1 inhibitor is selected from the group consisting of iadademstat, pulrodemstat, bomedemstat, seclidemstat, 1-((4-(methoxymethyl)-4-(((1R,2S)-2-phenylcyclopropylamino)methyl)piperidin-1-yl)methyl)cyclobutanecarboxylic acid, 3-(cyanomethyl)-3-(4-{[(1R,2S)-2-phenylcyclopropyl]amino}piperidin-1-yl)azetidine-1-sulfonamide, and pharmaceutically acceptable salts thereof.

24. The compound for use according to claim 20 or 21, wherein the LSD1 inhibitor is iadademstat or a pharmaceutically acceptable salt thereof.

25. The compound for use according to claim 24, wherein the LSD1 inhibitor is iadademstat dihydrochloride.

26. The combination product for use according to claim 19, the pharmaceutical composition for use according to claim 19, the article of manufacture for use according to claim 19, or the compound for use according to any one of claims 20 to 25, wherein the myeloid cancer is selected from acute myeloid leukemia and myelodysplastic syndrome.

27. The combination product for use according to claim 19, the pharmaceutical composition for use according to claim 19, the article of manufacture for use according to claim 19, or the compound for use according to any one of claims 20 to 25, wherein the myeloid cancer is acute myeloid leukemia.

28. The combination product for use according to claim 27, the pharmaceutical composition for use according to claim 27, the article of manufacture for use according to claim 27, or the compound for use according to claim 27, wherein the acute myeloid leukemia is relapsed or refractory acute myeloid leukemia.

29. The combination product for use according to claim 27 or 28, the pharmaceutical composition for use according to claim 27 or 28, the article of manufacture for use according to claim 27 or 28, or the compound for use according to claim 27 or 28, wherein the acute myeloid leukemia is acute myeloid leukemia with a FLT3 mutation.

30. The combination product for use according to claim 27, the pharmaceutical composition for use according to claim 27, the article of manufacture for use according to claim 27, or the compound for use according to claim 27, wherein the acute myeloid leukemia is relapsed or refractory acute myeloid leukemia with a FLT3 mutation.

31. A method for treating a myeloid cancer in a patient in need thereof, comprising administering to the patient a therapeutically effective amount of a combination product according to any one of claims 1 to 7 or a pharmaceutical composition according to any one of claims 8 to 12.

32. A method for treating a myeloid cancer in a patient in need thereof, comprising administering to the patient a therapeutically effective amount of an LSD1 inhibitor, or a pharmaceutically acceptable salt thereof, and a therapeutically effective amount of gilteritinib, or a pharmaceutically acceptable salt thereof.

33. The method according to claim 31 or 32, wherein the LSD1 inhibitor is a small molecule.

34. The method according to claim 31 or 32, wherein the LSD1 inhibitor is selected from the group consisting of iadademstat, pulrodemstat, bomedemstat, seclidemstat, 1-((4-(methoxymethyl)-4-(((1R,2S)-2-phenylcyclopropylamino)methyl)piperidin-1-yl)methyl)cyclobutanecarboxylic acid, 3-(cyanomethyl)-3-(4-{[(1R,2S)-2-phenylcyclopropyl]amino}piperidin-1-yl)azetidine-1-sulfonamide, and pharmaceutically acceptable salts thereof.

35. The method according to claim 31 or 32, wherein the LSD1 inhibitor is iadademstat or a pharmaceutically acceptable salt thereof.

36. The method according to claim 35, wherein the LSD1 inhibitor is iadademstat dihydrochloride.

37. The method according to any one of claims 31 to 36, wherein the myeloid cancer is selected from acute myeloid leukemia and myelodysplastic syndrome.

38. The method according to any one of claims 31 to 36, wherein the myeloid cancer is acute myeloid leukemia.

39. The method according to claim 38, wherein the acute myeloid leukemia is relapsed or refractory acute myeloid leukemia.

40. The method according to claim 38 or 39, wherein the acute myeloid leukemia is acute myeloid leukemia with a FLT3 mutation.

41. The method according to claim 38, wherein the acute myeloid leukemia is relapsed or refractory acute myeloid leukemia with a FLT3 mutation.

42. The method according to any one of claims 31 to 41, wherein the patient to be treated is a human.

43. The method according to any one of claims 31 to 42, wherein the LSD1 inhibitor or the pharmaceutically acceptable salt thereof and gilteritinib or the pharmaceutically acceptable salt thereof are administered in the same pharmaceutical formulation.

44. The method according to any one of claims 31 to 42, wherein the LSD1 inhibitor or the pharmaceutically acceptable salt thereof and gilteritinib or the pharmaceutically acceptable salt thereof are administered in separate pharmaceutical formulations.

45. Use of a combination comprising an LSD1 inhibitor or a pharmaceutically acceptable salt thereof and gilteritinib or a pharmaceutically acceptable salt thereof for the manufacture of a medicament for the treatment of a myeloid cancer.

46. Use of an LSD1 inhibitor or a pharmaceutically acceptable salt thereof for the manufacture of a medicament for the treatment of a myeloid cancer, to be used in combination with gilteritinib or a pharmaceutically acceptable salt thereof.

47. Use of gilteritinib or a pharmaceutically acceptable salt thereof for the manufacture of a medicament for the treatment of a myeloid cancer, to be used in combination with an LSD1 inhibitor or a pharmaceutically acceptable salt thereof.

48. Use of a combination comprising an LSD1 inhibitor or a pharmaceutically acceptable salt thereof and gilteritinib or a pharmaceutically acceptable salt thereof for the treatment of a myeloid cancer.

49. Use of an LSD1 inhibitor or a pharmaceutically acceptable salt thereof for the treatment of a myeloid cancer, to be used in combination with gilteritinib or a pharmaceutically acceptable salt thereof.

50. Use of gilteritinib or a pharmaceutically acceptable salt thereof for the treatment of a myeloid cancer, to be used in combination with an LSD1 inhibitor or a pharmaceutically acceptable salt thereof.

51. The use according to any one of claims 45 to 50, wherein the LSD1 inhibitor is a small molecule.

52. The use according to any one of claims 45 to 50, wherein the LSD1 inhibitor is selected from the group consisting of iadademstat, pulrodemstat, bomedemstat, seclidemstat, 1-((4-(methoxymethyl)-4-(((1R,2S)-2-phenylcyclopropylamino)methyl)piperidin-1-yl)methyl)cyclobutanecarboxylic acid, 3-(cyanomethyl)-3-(4-{[(1R,2S)-2-phenylcyclopropyl]amino}piperidin-1-yl)azetidine-1-sulfonamide, and pharmaceutically acceptable salts thereof.

53. The use according to any one of claims 45 to 50, wherein the LSD1 inhibitor is iadademstat or a pharmaceutically acceptable salt thereof.

54. The use according to claim 53, wherein the LSD1 inhibitor is iadademstat dihydrochloride.

55. The use according to any one of claims 45 to 54, wherein the myeloid cancer is selected from acute myeloid leukemia and myelodysplastic syndrome.

56. The use according to any one of claims 45 to 55, wherein the myeloid cancer is acute myeloid leukemia.

57. The use according to claim 56, wherein the acute myeloid leukemia is relapsed or refractory acute myeloid leukemia.

58. The use according to claim 56 or 57, wherein the acute myeloid leukemia is acute myeloid leukemia with a FLT3 mutation.

59. The use according to claim 56, wherein the acute myeloid leukemia is relapsed or refractory acute myeloid leukemia with a FLT3 mutation.

60. The combination product for use according to any one of claims 19 or 26 to 30, the article of manufacture for use according to claims 19 or 26 to 30, the compound for use according to any one of claims 20 to 30, the method according to any one of claims 31 to 44, or the use according to any one of claims 45 to 59, wherein the LSD1 inhibitor or the pharmaceutically acceptable salt thereof and gilteritinib or the pharmaceutically acceptable salt thereof are administered orally.

61. The combination product for use according to any one of claim 19, 26 to 30 or 60, the article of manufacture for use according to claim 19, 26 to 30 or 60, the compound for use according to any one of claim 20 to 30 or 60, the method according to any one of claim 31 to 42 or 60, or the use according to any one of claims 45 to 60, wherein the LSD1 inhibitor or the pharmaceutically acceptable salt thereof and gilteritinib or the pharmaceutically acceptable salt thereof are administered using separate pharmaceutical formulations.

62. The combination product for use according to claim 61, the article of manufacture for use according to claim 61, the compound for use according to claim 61, the method according to claim 61, or the use according to claim 61, wherein the LSD1 inhibitor or the pharmaceutically acceptable salt thereof and gilteritinib or the pharmaceutically acceptable salt thereof are administered simultaneously using separate pharmaceutical formulations.

63. The combination product for use according to claim 61, the article of manufacture for use according to claim 61, the compound for use according to claim 61, the method according to claim 61, or the use according to claim 61, wherein the LSD1 inhibitor or the pharmaceutically acceptable salt thereof and gilteritinib or the pharmaceutically acceptable salt thereof are administered sequentially using separate pharmaceutical formulations.
